(19) United States
(12) Patent Application Publication
(10) Pub. No.: US 2019/0001016 A1
(43) Pub. Date: Jan. 3, 2019

(54) BANDAGE

(71) Applicant: Karl Otto Braun GmbH & Co. KG, Wolfstein (DE)

(72) Inventor: Guenter Langen, Wolfstein (DE)

(21) Appl. No.: 16/064,590

(22) PCT Filed: Dec. 23, 2016
(86) PCT No.: PCT/EP2016/082627

§ 371 Date: Jun. 21, 2018

(30) Foreign Application Priority Data
Dec. 23, 2015 (DE) 10 2015 226 645.1

(51) Int. Cl. A61L 15/12; A61F 13/02

(52) U.S. Cl. CPC A61L 15/12; A61F 2013/00187; A61F 13/0283; A61F 13/0273

(57) ABSTRACT

The invention relates to a bandage comprising a flat web material as a substrate, wherein a self-adhesive cohesive adhesive compound is at least partially applied to both flat sides of the substrate, and the substrate is impregnated with a liquid preparation, characterised in that said adhesive compound comprises an anhydrous cohesive pressure sensitive adhesive which contains natural and/or synthetic rubber, this natural and/or synthetic rubber being dissolved in an organic solvent for application, or the pressure sensitive adhesive being molten for application.

The invention relates to a bandage comprising a flat-web material as substrate, wherein a self-adhesive cohesive adhesive has been at least partially applied on both flat sides of the substrate and the substrate has been impregnated with a liquid preparation. In the prior art, cohesive adhesive bandages containing liquid preparations have been known for many years, for example as cold bandages. For this purpose, a dry bandage is impregnated with a cooling fluid based on, for example, water/ethanol or water/isopropanol, these mixtures having been optionally admixed with further auxiliaries. Such products are, for example, known under the brand names “Sporty Cool” from Karl Otto Braun GmbH & Co. KG, Wolfstein, Germany and under “Ideal Cool” from Paul Hartmann AG, Heidenheim, Germany. Said products use as substrate an elastic textile material which has been coated with natural rubber latex in the form of the aqueous dispersion as cohesive pressure-sensitive adhesive. When dry, the bandage is then impregnated with the cooling liquid. Essential oils can, for example, be used as auxiliaries.

Such bandages are used as compression bandages and/or support bandages for use in blunt injuries, including in sports medicine. The coldness and the compression counteract swellings.

Furthermore, it is, for example, known from DE 295 18 881 U1 to provide a compression bandage designed to be both adhesive and cooling. The bandage is intended to be a bandage which cools a body part without chemical processes taking place and without precooling in a refrigerator or freezer. To this end, the bandage comprises a silicone-based gel and is, furthermore, designed to be adhesive. To provide the cooling action, it must be immersed in water every four to six hours in order to maintain the cooling action.

In addition, WO 2005/007050 A1 describes a cold bandage based on an elastic support material containing a cooling liquid preparation based on ethanol, water, methanol and isoamyl acetate.

A disadvantage of the aforementioned bandages formed on the basis of natural rubber latex with regard to their cohesive adhesive forces is that the cohesive forces lessen during the storage time, with the result that it is no longer possible to reliably realize a secure layer adhesion of the wet bandage over a wearing time of up to several days, as is desirable.

It is therefore an object of the invention to provide a cohesive self-adhesive cold bandage which is aging-stable, with the result that its complete functionality with respect to the adhesion properties, but also to the cooling action, is maintained even with a storage time of several months.

The invention therefore achieves the object by a bandage having the features of claim 1 and a method having the features of claim 11, wherein the bandage is designed such that the adhesive comprises an anhydrous cohesive pressure-sensitive adhesive comprising natural rubber and/or synthetic rubber, in which the natural rubber and/or synthetic rubber has been dissolved in an organic solvent for the purposes of application or the pressure-sensitive adhesive is a melted liquid for the purposes of application.

In this way, it is possible to use a rubber material for the cohesive properties without having to resort to a latex material.

In this connection, the use of a cohesive pressure-sensitive adhesive based on natural rubber or synthetic rubber, synthetic rubber being preferred, is able to provide a cohesive coating material for a bandage that, surprisingly, exhibits an aging behavior different from natural rubber latex and thus still provides a satisfactory adhesive force even after relatively long storage, even for wet bandages. The adhesive force between the layers of a dry bandage according to the invention corresponds virtually identically to the adhesive force of bandages based on natural rubber latex. If, then, both the bandage according to the invention and the bandage according to the prior art are impregnated with a liquid preparation, composed of 50% ethanol in water for example, the layers of the bandage according to the invention, surprisingly, still adhere to one another, this continuing to be the case after a storage time of several months for the impregnated bandage, whereas the bandage according to the prior art does not adhere or only adheres minimally. Therefore, the bandage according to the invention has the advantage that there is a secure layer adhesion of the wet bandage without use of bandage clips over a wearing time of up to several days, even if the bandage was already stored for several months.

In this connection, it is possible to envisage according to the invention that the natural rubber (polyisoprene) or preferably the synthetic rubber (polyisoprene, polybutadiene, polychloroprene) is realized as an open-pore coating, preferably only partially, in the form of fragments or patterns.

In this connection, an appropriate application can be carried out in various ways, namely, for example, a) in the form of a spray application of solutions of the solid rubber in organic solvents, it being possible to use here C₅- to C₁₂-hydrocarbons, ethers, esters or ketones, and additionally b) a spray application of melt adhesives.

Furthermore, appropriate solutions of the rubber material in organic solvents can also be processed in the context of stencil printing, it alternatively also being possible to apply the melt adhesives in the stencil printing method. Furthermore, anilox roll application is also conceivable both for the solutions and for the melt adhesives.

Further application methods are possible, doctor blades or knife-over-rolls for the rubber solutions as well as casting methods (curtain coating) for the rubber solutions.

In this connection, the aforementioned methods are to be carried out such that there is a microporous, open-pore application of rubber fragments in an application quantity per surface area of 2 to 20 g/m² after coating, drying and cooling, wherein the rubber fragments are arranged preferably on the surface and can be bonded adhesively to the substrate, specifically, in this case, especially via an adherence to the fibers.

The cohesive pressure-sensitive adhesive achieves a nonslip fit in the case of wrapping on the body part.

If the rubber is introduced into solution with organic solvents, the fraction thereof is 5 to 20% solids content. The applied rubber solution is then treated in a drying step such that the solvent evaporates, with the result that a closed or open-pore rubber film then remains on the substrate surface after the drying process. Upon pressing, said rubber film will seal to itself or adheres together, thereby providing the cohesive adhesive force.

In this connection, the bandage can be elastic in the longitudinal direction and/or transverse direction. In this case, it can be envisaged in particular that the substrate is a woven fabric, warp-knitted fabric, weft-knitted fabric or nonwoven. The elasticity can, in this case, be produced by the incorporation of elastic threads, such as, for example, high-twist cotton threads, as spun or twisted crepe threads, through textured polyamide or polyester yarns as well as rubber or polyurethane threads or combinations thereof. For example, using the stitch-bonding technique MALIWATT, elasticity can be obtained by stitch-over of a rigid nonwoven with permanently elastic elastane threads in the longitudinal direction. The stitch-bonding technique MALIWATT is described in Malimo Nähwirktechnologie [Malimo stitch-bonding technology], Ploch, Böttcher, Scharch, VEB Fachbuchverlag Leipzig, 1978, 1st edition.

The liquid preparation according to the invention consists of a single-phase or multiphase preparation based on water with additions of inorganic substances, such as, for example, salts, or organic substances, namely, for example, alcohols, esters, ethers, ketones or terpenes. However, it is also possible to use gels or foams as preparations. As a result, defined physical and physiological actions are exerted on the body, such as, for example, cooling, heating, moistening, hydration, delipidation, relipidation, pain relief or stimulus inhibition, such as, for example, inhibition of itching.

The quantity of fluid that is required for the intended action is applied to the cohesive bandage after application of the adhesive by spraying, immersion, padding, slop-padding and/or casting. The preparation can be distributed in a partial manner or over the entire bandage.

To avoid losses due to drying as a result of evaporation or losses of active ingredient, the bandage can then be introduced into an item of packaging that is hermetically sealed, such as, for example, film bags, peel packs, thermoformed packs, containers, bottles, etc., and a storage time of up to 3 years can thus be achieved.

A bandage manufactured in this way has a cohesive self-adhesion both in the wet state and in the dry state, which self-adhesion is still present even after a storage process of several years.

The invention shall be more particularly elucidated below on the basis of examples.

### EXAMPLE 1

### Cohesive Nonwoven Bandage Containing Cooling Fluid

The web substrate used is an elastic nonwoven construction type 752, brand name NOWOPRESS 752, manufacturer Karl Otto Braun GmbH & Co., Wolfstein, Germany, wherein the base textile was obtained using the stitch-bonding technique MALIWATT by stitch-over of a rigid polypropylene nonwoven with permanently elastic elastane threads in the longitudinal direction. For details about this support textile, see the table below:

| | |
|---|---|
| Material composition of | 84% polypropylene, 16% |
| elast. nonwoven type 752 | elastane |
| Base nonwoven | PP spunbond nonwoven, |
| | 35 g/m², thermally embossed, |
| | color blue (Pantone 18-1662 |
| | TPX) |
| Sewing thread | 133 dtex elastane |
| | (Dorlastan, manufacturer |
| | ASAHI Kasai, Dormagen, |
| | Germany) |
| Sewing thread density | 45 threads per 10 cm width |
| Sewing thread stitch length, | 3 mm, open pillar stitch |
| stitching | |
| Elasticity | Longitudinal direction (warp |
| | direction) |
| Stretchability in accordance | 220% |
| with DIN 61632 at F = 3 N/cm | |

This elastic base textile is, as web material of width 10 cm, coated on both surfaces with a cohesive pressure-sensitive adhesive based on polyisoprene rubber using a knife-over-roll in order to achieve the desired cohesive adhesive effect. The cohesive pressure-sensitive adhesive consists of the following components:

1. Solid rubber: cis-1,4-polyisoprene, brand name NATSYN 2200, manufacturer: GOODYEAR, Ohio, USA

2. Solvent: isohexane, brand name ISOHEXAN, C6 hydrocarbon mixture of various isomers, manufacturer: BIESTERFELD, Hamburg, Germany

3. Antiaging agent: 2,6-di-tert-butyl-p-cresol, brand name IONOL CP, manufacturer: OXIRIS Chemicals, Sant Celoni, Spain

Production is carried out according to the following instructions: 400 g of isohexane (component 2) are initially charged in a stainless-steel stirring tank and altogether 60.6 g of solid rubber (component 1) are added under stirring in the form of granular material with a particle size of approximately 4 mm. 1.21 g of antiaging agent (component 3) are likewise added as powder. At a stirring speed of 300 rpm, component 3 dissolves immediately in the isohexane, whereas approximately 120 minutes are required for the complete dissolution of component 2. The result is a grayly turbid viscous adhesive having a solids content of 13.1%. The viscosity was measured using a Haake viscometer, model “Haake Viscotester VT 2 plus”, manufacturer: THERMO SCIENTIFIC FISHER, Braunschweig, Germany, at T=20° C. at 12 500 mPas.

The elastic nonwoven type 752 is coated in such a way that the 10 cm wide web is fed to a knife-over-roll unit at a transport speed of 5 m/min, with use of lowest possible tensile forces in order to largely maintain the unstretched state of the web type 752. The width of the knife-over-roll is 15 cm. The material web is guided above the roller and below the doctor blade and the pasty adhesive is charged in portions before the doctor blade, with the result that there is always a quantity of approximately 100 g of adhesive on the material web. The gap between doctor blade and roller is adjusted such that the wet application of the adhesive is between 55 and 65 g/m² under stretching (in accordance with DIN 61632 at 3 N/cm). Directly after the knife-over-roll, the adhesive-coated material web passes through a drying channel, length 3.0 m, in which the solvent (isohexane) is evaporated from the adhesive with a hot-air stream at T=80° C. The solid adhesive forms a microporous, open-pore film of rubber fragments. The web material leaves the dryer in the dry state and is wound up under low tension to form a roll. The web material is now coated on one side with the dried adhesive; the application quantity is between 7.2 and 8.5 g dry weight. The areal weight of the web material coated on one side is 50.2 to 51.5 g and the stretchability is approximately 190% (both parameters in accordance with DIN 61632 at 3 N/cm). The roll of the web material coated on one side now passes through the knife-over-roll unit and the drying channel, as described above, in a second pass. In this case, the hitherto uncoated side of the substrate is coated with the adhesive in an analogous operation. The web material now coated on both sides leaves the dryer in the dried state and is wound up under low tension to form a roll. The solid adhesive forms a microporous, open-pore film of rubber fragments on both surfaces of the substrate. To avoid the layers of the webs sticking together via the adhesive rubber surface, a separating paper or a separating film is fed as spacer when winding the roll and said paper/film is wrapped up between the layers. The application quantity of the adhesive on the substrate is then altogether between 14.4 and 17.0 g dry weight. The areal weight of the web material coated on both sides is 57.4 to 60.0 g and the stretchability is approximately 160% (both parameters in accordance with DIN 61632 at 3 N/cm).

A winding core, for example a plastic tube composed of polypropylene, inner diameter 28 mm, wall thickness 1 mm, height 10.0 cm, is then used to wind a roll length of 1.75 m web material under low tension to form a cylindrical spool body, which is the starting material for the production of the fluid bandage according to the invention. The net weight (web material without tube) is 27.0 g. The bandage diameter is 54 mm.

Mixtures of ethanol/water with or without addition of cooling auxiliaries are used as cooling fluid:

Cooling fluid KF1: Produced by mixing the following components:
48 g of ethanol/water mixture (containing 96% by volume of ethanol)
50 g of water, demineralized
2.0 g of Eumulgin HRE 40 (emulsifier, manufacturer COGNIS, Düsseldorf, Germany)

Cooling fluid KF2 (with enhanced cooling action):
Mixture/dissolution of the following components
45 g of ethanol/water mixture (containing 96% by volume of ethanol)
50 g of water, demineralized
0.05 g of camphor
0.05 g of menthol
0.20 g of menthyl lactate
4.70 g of Eumulgin HRE 40 (emulsifier, manufacturer COGNIS, Düsseldorf, Germany)

A first cold bandage, type KBF1, is produced by placing a spool body in a plastic container with screw-on lid, inner diameter 67 mm, height 110 mm, and slowly pouring 40.0 g of cooling fluid KF1 over it until the fluid is completely absorbed by the bandage. The fluid is homogeneously distributed in the bandage within a minute. A second cold bandage, type KBF2, is produced by placing a spool body in a plastic container with screw-on lid, inner diameter 67 mm, height 110 mm, and slowly pouring 40.0 g of cooling fluid KF2 over it until the fluid is completely absorbed by the bandage. The fluid is homogeneously distributed in the bandage within a minute.

After a waiting time of 24 hours, the bandages were subjected to a physical test and the following results were ascertained:

| Cold bandage test results | Type KBF1 | | Type KBF2 | |
|---|---|---|---|---|
| Weight of fluid bandage (without | 67.0 | g | 67.0 | g |
| tube) | | | | |
| Stetchability (DIN 61632 at 3 N/cm) | 152% | | 158% | |
| Unwinding force (cN/cm) | 35.8 | cN/cm | 47.6 | cN/cm |
| Adhesive force, side A/B | 22.5 | cN/cm | 20.8 | cN/cm |
| ΔCTₘₐₓ = Max. cooling | −4.2 | K. | −5.5 | K. |
| temperature difference | | | | |

The cooling temperature difference ACT is ascertained in an application test on a test subject as follows: the cold bandage is wound on the lower arm of the test subject at a 5-10 cm distance from the elbow as a 2-layer ring which overlaps to form straight edges, and is lightly pressed in order to bond the layers. A digital thermometer with metal sensor tip is then immediately pushed under the bandage so that the tip is centrally positioned. Similarly, the starting temperature of the skin T(S) was ascertained beforehand using a dry ideal bandage in accordance with DIN 61632. The skin temperature under the cold bandage T(B) is measured in intervals of 1 minute (initially) to 15 minutes for a period from t=0 to maximally t=180 minutes. The cooling temperature difference ACT is calculated as the difference T(B)−T(S). ACTmax is the maximum cooling temperature difference, i.e. the value T(B)−T(S) is maximal.

In relation to use, the bandages are wound on the lower leg as a spiral bandage under slight stretching of approximately 80% with 50% overlap and the layers are firmly pressed by hand. Surprisingly, the layers form an adhesive bond, even though the cooling fluid also covers the rubber fragments of the cohesive coating. The test subject feels the cooling action immediately after application of the bandages, with the peak of the cooling action being reached after 30-60 minutes. The cooling action persists for altogether approximately 90 to 120 minutes. Subjectively, the test subjects feel a higher cooling action with the bandage KBF2 than with the bandage KBF1.

Furthermore, the invention also provides a method for producing a bandage comprising a flat-material web as substrate, wherein a self-adhesive cohesive adhesive has been at least partially applied on both flat sides of the substrate, involving applying the adhesive in the form of an anhydrous cohesive pressure-sensitive adhesive comprising natural rubber and/or synthetic rubber, in which pressure-sensitive adhesive the natural rubber and/or synthetic rubber has been dissolved in an organic solvent for the purposes of application or is a melted liquid for the purposes of application, and then either evaporating the solvent or curing the pressure-sensitive adhesive and then impregnating the substrate coated with the cohesive adhesive with a liquid preparation.

### EXAMPLE 2

### Cohesive Web Bandage Containing Aluminum Acetotartrate

The web substrate used is an elastic woven-fabric construction type 199, manufacturer: Karl Otto Braun GmbH & Co. KG, Wolfstein, Germany, wherein the elasticity of the base textile in the warp direction results from the combination of permanently elastic elastane threads with rigid cotton yarns. For details about this support textile, see the table below:

| | |
|---|---|
| Material composition of | 99% cotton, 1% elastane |
| woven fabric type 199 | |
| Warp thread-material | Warp thread A: 20 tex cotton |
| | Warp thread B: 15.6 tex |
| | lycra (elastane) surrounded |
| | by 24 tex cotton |
| Warp order | 4 threads A-1 thread B |
| Thread coun/warp density | 110 per 10 cm width |
| Weft thread-material | 50 tex cotton |
| Thread count/weft density | 130 per 10 cm stretched |
| | (DIN 61632) |
| Areal weight, stretched | 95 g/m² |
| Elasticity | In the longitudinal |
| direction (warp direction) | |
| Stretchability/retraction | 120%/99% |
| in accordance with | |
| DIN 61632 | |

The elastic woven fabric type 199 is coated using the adhesive described in Example 1 on the same knife-over-roll coating machine, using a 10 cm wide web. The application quantity of the adhesive on the substrate is then altogether 13.0 g dry weight. The areal weight of the web material coated on both sides is 108 g and the stretchability is 100% (both parameters in accordance with DIN 61632 at 3 N/cm).

A winding core, for example a plastic tube composed of polypropylene, inner diameter 28 mm, wall thickness 1 mm, height 10.0 cm, is used to wind a length of 2.0 m web material of the woven fabric type 199 under low tension to form a cylindrical spool body, which is the starting material for the production of the fluid-impregnated bandage according to the invention.

The net weight (web material without tube) is 44.0 g. The bandage diameter is 57 mm.

The fluid used is a dilute aqueous solution of aluminum acetotartrate (standardized, see Deutsches Arzneimittelbuch [German pharmacopoeia], 2012 edition (DAB 2012)). This active ingredient has an antiseptic, astringent and antiswelling action and is, for example, used in contusions.

Fluid KF3: Produced by mixing the following components:
10 g of aluminum acetotartrate solution as per DAB 2012
90 g of water, demineralized
2.0 g of Eumulgin HRE 40 (emulsifier, manufacturer COGNIS, Düsseldorf, Germany)

The fluid bandage (KBF3) according to Example 2 is produced by placing a spool body in a plastic container with screw-on lid, inner diameter 67 mm, height 110 mm, and slowly pouring 44.0 g of fluid KF3 over it until the fluid is completely absorbed by the bandage. The fluid is homogeneously distributed in the bandage within a minute.

After a waiting time of 24 hours, the bandages were subjected to a physical test and the following results were ascertained:

| Cold bandage test results | Type KBF3 | |
|---|---|---|
| Weight of fluid bandage | 88.0 | g |
| (without tube) | | |
| Stetchability (DIN 61632 at | 99% | |
| 3 N/cm) | | |
| Unwinding force (cN/cm) | 72.7 | cN/cm |
| Adhesive force, side A/B | 28.5 | cN/cm |

Concerning use, the bandages are wound on the lower leg as a spiral bandage under slight stretching of approximately 60% with 50% overlap and the layers are firmly pressed by hand. Surprisingly, the layers form an adhesive bond, even though the fluid composed of aluminum acetotartrate solution forms an interface layer between the rubber fragments of the cohesive coating. The test subject feels a pleasantly cooling and skin-drying (astringent) action. Swellings recede.

1. A bandage comprising a flat-web material as substrate, wherein a self-adhesive cohesive adhesive has been at least partially applied on both flat sides of the substrate and the substrate has been impregnated with a liquid preparation, and wherein the self-adhesive cohesive adhesive comprises either an anhydrous cohesive pressure-sensitive adhesive comprising natural rubber and/or synthetic rubber, in which the natural rubber and/or synthetic rubber has been dissolved in an organic solvent for the purposes of application or an anhydrous cohesive pressure-sensitive adhesive which is a melted liquid for the purposes of application.

2. The bandage of claim 1, wherein the anhydrous cohesive pressure-sensitive adhesive which is a melted liquid for the purposes of application comprises natural rubber and/or synthetic rubber.

3. The bandage of claim 1, wherein the bandage is elastic in the longitudinal direction and/or transverse direction.

4. The bandage of claim 1, wherein the liquid preparation is selected from the group consisting of a single-phase solution, a multiphase emulsion, a gel and a foam.

5. The bandage of claim 4, wherein the liquid preparation is a single-phase solution based on a multiphase emulsion based on water with an addition of an inorganic substance, or an organic substance.

6. The bandage of claim 1, wherein the substrate is selected from the group consisting of a woven fabric, a warp-knitted fabric, a weft-knitted fabric and a nonwoven fabric.

7. The bandage of claim 1, wherein the substrate comprises elastic threads, spun or twisted crepe threads, rubber and/or polyurethane elastane threads and combinations thereof.

8. The bandage of claim 1, wherein the application of the self-adhesive cohesive adhesive results in a cohesive coating which is open-pore.

9. The bandage of claim 1, wherein the adhesive has been applied in patterns.

10. The bandage of claim 1, wherein the application of the adhesive is 2 to 20 g/m².

11. A method for producing the bandage of claim 1, wherein a substrate in the form of a flat-web material is at least partially coated on both sides with a cohesively self-adhesive pressure-sensitive adhesive and the flat-web material is then converted to bandages and the bandage is then impregnated with a liquid preparation.

12. The method of claim 10, wherein the cohesive adhesive is applied first to one flat side of the flat-web material and then to the second flat side of said flat-web material.

13. The bandage of claim 5, wherein the inorganic substance is a salt.

14. The bandage of claim 5, wherein the organic substance is selected from the group consisting of alcohols, esters, ethers, ketones, and terpenes.

15. The bandage of claim 7, wherein the elastic threads are high twist cotton threads.

16. The bandage of claim 7, wherein the spun or twisted crepe threads are textured polyamide or polyester yarns.

17. The method of claim 11, wherein the substrate is an elastic substrate.
